(19) United States
(12) Patent Application Publication
(10) Pub. No.: US 2019/0110917 A1
(43) Pub. Date: Apr. 18, 2019

(54) FOOT PLATE AND ORTHOTIC

(71) Applicant: OTTOBOCK SE & CO. KGAA, Duderstadt (DE)

(72) Inventors: Gordon SIEWERT, Gottingen (DE); Markus TUTTEMANN, Waltrop (DE); Marcus LURSSEN, Gottingen (DE)

(73) Assignee: OTTOBOCK SE & CO. KGAA, Duderstadt (DE)

(21) Appl. No.: 16/097,244

(22) PCT Filed: Apr. 26, 2017
(86) PCT No.: PCT/EP2017/059866

§ 371 Date: Oct. 27, 2018

(30) Foreign Application Priority Data
Apr. 29, 2016 (DE) 10 2016 108 055.1

(51) Int. Cl. A61F 5/01; A43B 13/22; A61F 5/14

(52) U.S. Cl. CPC A61F 5/0111; A61F 5/14; A61F 5/0127; A43B 13/223

(57) ABSTRACT

A foot plate for supporting a foot, wherein the foot plate has a front edge and at least one weakening which is spaced apart from the front edge and extends in the medial-lateral direction of the foot plate, a weakening on the underside having an orientation or rounded contour directed obliquely downwards from anterior to posterior, and a weakening on the upper side having an orientation or rounded contour directed obliquely upwards from anterior to posterior.

The invention relates to a foot plate and an orthotic with a foot plate for supporting a foot with a foot support protruding from it in the proximal direction and a lower leg support bar to be applied to a lower leg, whereby the foot plate has a front edge.

Foot plates are used to support a foot, for example in a shoe as an insert or as a support for orthotics. They may be made from flexible material or a combination of materials. They may also be elastic and/or have a stability or rigidity that makes it possible to support a foot resting on a foot plate and, if necessary, to transfer and absorb the necessary forces and momentum that occur when the foot plate is used.

Orthotics are technical orthopedic devices that are worn on the body, for example on an extremity or on the torso and that are attached to the respective body part by means of attachment elements such as belts, sleeves, shoes or clamps. The attachment elements may be closed for example with snaps or hook-and-loop fasteners to obtain a fit with the respective extremity that is customized to the person wearing the orthotic. Orthotics are generally used to support, stabilize, brace, relieve, or even to restrict the movement of the respective extremities or joints. They are also used to correct defective positions, to correctly establish or maintain a particular orientation, and to protect joints and extremities.

In the area of the lower extremities, orthotics are formed for example as knee/ankle/foot orthotics (KAFO) or as ankle/foot orthotics (AFO) and may be used to reduce the effect of dorsiflexion and paralysis. To this purpose, a spring-loaded joint may be provided in the area of the natural ankle that presses the foot plate against the lower leg support bar to perform a dorsal flexion in the swing phase so that it is still possible for the foot to move without the toes or tip of the foot dragging on the floor. Instead of a spring-loaded joint, it is also possible to connect a spring of a foot plate with a lower leg support bar.

An orthotic to correct a defective leg position is known from EP 2 563 300 B1 comprising a contact base that reaches under the foot of a person and establishes contact with a walking surface, i.e., either a sole or a floor. The orthotic has a support bar arrangement that extends in an upper lateral direction, which can be secured to the person's lower leg by means of a securing device, whereby a rigid elbow is formed under load at the transition from the contact base to the support bar arrangement. The support bar arrangement is divided by a swivel joint, which is approximately the same height as the natural ankle, into a piece that is to be applied to the side of the foot and a support bar that is applied to the side of the lower leg. The support bar is designed as a spring element. By means of the torque resulting from a preset tension of the spring element relative to the lower leg, a laterally acting corrective force is applied to the lower leg.

US 2014/0316316 A1 pertains to a total contact cast with a foot pad from semi-rigid plastic and a foam pad applied on top of it. Extending from the foot pad in a vertical direction are two lower leg support bars. In the horizontal direction of the foot pad, several notches are formed across the entire width of its underside.

U.S. Pat. No. 8,574,181 B2 pertains to a walking aid used for injuries in the ankle area. The underside of a lining of the walking aid comprises a track and two connecting passages that serve to attach a sole with the corresponding connecting elements on their upper side and with a walking area profile on their underside.

EP 619 99 A1 pertains to a lower leg orthotic with a sole that comprises an outward-bending profile on its opposing ends, in which a plurality of recesses is formed across the entire width of the sole. A heel pad that can be cut and two ankle pads that can be cut are inserted between the heel and the orthotic as well as the ankle and the orthotic to improve the fit.

The respective foot plates or support bases are either customized or have a standard size that is sized in such a way that the patients in question can use them without any problems. To adapt the foot plate or the support base to the respective shoe or the respective patient, these foot plates are adapted, shortened, their form modified or ground down by an orthopedic mechanic. With regard to inserts for shoes, markings are applied to the sole of the insert along which a shortening or contour change can be made with a scissor so that the standard shapes of a shoe are covered.

It is the task of the present invention to provide a foot plate and an orthotic with which an easier adaptation to different shoe shapes and/or patients can be made.

According to the invention, this task is solved by a foot plate with the features of the main claim and an orthotic with the features of the coordinate claim. Preferred embodiments and further developments of the invention are disclosed in the respective subclaims, the description and the figures.

The foot plate and an orthotic with such a foot plate to rest the foot on with a foot support protruding from it in the proximal direction and a lower leg support bar to be applied to a lower leg, whereby the foot plate has a front edge, provide that the foot plate has at least one weakening which extends in the medial-lateral direction, wherein the weakening is spaced apart from the front edge. This weakening spaced apart from the front edge makes it possible to make an easy adjustment of the contour or length of the foot plate along the weakening so that an easier customization of the foot plate, for example as an insert and/or the orthotic can be performed. To this purpose, the foot plate is cut along the respective weakening and the material in front of the weakening removed. This way, both the form and the length of the foot plate can be adapted to the respective user and/or the shoe in which the foot plate or the orthotic is to be worn. A weakening arranged on the underside of the foot plate has an orientation directed obliquely downward from anterior to posterior so that front edge of the foot plate can, after having been shortened or having adapted the contour, has a straight or rounded orientation directed obliquely downward from anterior to posterior which makes it easier to roll the foot along the foot plate. If the weakening is arranged at the upper side of the foot plate, the weakening has an orientation directed obliquely upward from the anterior to the posterior. From the other point of view, in the case of a weakening on the upper side with a substantially smooth underside, the weakening has an orientation in the direction of the underside from posterior to anterior. In the case of a weakening on the underside with a smooth surface, the weakening has an orientation from the underside from the anterior to the posterior in the direction of the upper side. Furthermore, an oblique or rounded front edge of an adapted orthotic, even in the case of a rounded contour with a convexity, the front edge is considered less bothersome than if it were to have an orientation that runs vertical to the underside or the upper side. Preferably, the front edge of the weakening after the shortening or adaptation of the contour ends in a material thickness that is less than 2 mm, preferably 0.5 mm.

The weakening may be continuous so that it is easy to adapt along the weakening that is generally formed as a line. It is also possible that the weakening is formed as a broken line or perforation. A weakening as a continuous line has the advantage of a uniform shape of the outer edge, which leads to better comfort and no, or just some, work on the remaining foot plate to smoothen the new front edge.

The weakening may extend across the entire width of the foot plate, whereby in particular a shortening of the foot plate and an adaptation to the wishes and particulars of the respective foot plate or orthotic user could be performed.

Preferably, the weakening or weakenings follow the contour of the front edge of the initial shape of the foot plate or orthotic so that the overall shape is maintained and so that only the length of the foot plate is adapted. Alternatively, every weakening may have a contour or form and/or course that deviates from the weakening arranged before or behind it so that not only the length but also another shape can be obtained depending on the length.

In a further development, the weakening is arranged behind or posterior to a metatarsal joint, because the foot plate is not perceived as bothersome due to the material reduction in the area of the weakening. If an end edge exists in front of the metatarsal joint, this is not perceived as less comfortable so that a tapering of the foot plate in or behind the metatarsal joint is preferred.

A plurality of weakenings may be successively arranged at equal distances, i.e., from the anterior direction in the posterior direction of the foot plate, so that a plurality of different, preset lengths and/or shapes can be selected which makes the foot plate and/or the orthotic adaptable to many foot sizes or foot shapes or shoe sizes or shoe shapes.

The weakening may have the form of a slit, recess, and/or reduction in the material of the foot plate. A slit or a plurality of slits consecutively arranged in a line make it easy to preset the desired front contour that is finalized by a deepening of the slit across the entire material strength of the foot plate. Equally, the weakening may be formed as a recess, for example as a material ablation subsequently applied by grinding, milling or other separation methods, in particular on the underside of the foot plate. Weakenings may be integrated in the foot plate in the form by reductions in material as well that may be incorporated already when the foot plate is produced.

A further development of the invention provides that material is arranged or is arrangeable in the weakening and fills the weakening. The additional material may differ from a base material of the foot plate or be made from the same material. Since the weakening has, for example, the form of a notch or a reduction in material in the foot plate's base material, the weakening may be filled or filled up with material to make the surface of the foot plate, and in particular the underside of the foot plate, more even. Said material may have a different color or only be partially attached to the base material of the foot plate. The material inserted into the weakening may be adhered in a detachable manner. It may have a smooth or continuous surface so that the user perceives the foot plate as smooth or even. In the additional material, perforation lines may be formed along which the excess material can be torn off when the foot plate is shortened. Alternatively, the filler material may, after the adaptation and shortening of the foot plate, be inserted, for example glued, jammed, or pushed, into the remaining weakening. A filling or at least partial filling of the weakenings is particularly advantageous when the weakenings are arranged on the upper side. When the weakenings are formed so that they face the foot, a filling is advantageous for reasons of comfort.

The deviation of the additional material, which may deviate from the base material, may therefore differ in terms of its color or in terms of the mechanical properties of the material. If the contour of the foot plate is changed along the weakening, the different material is completely removed from the respective weakening so that the contour of the weakening then provides the outer contour of the foot plate after the material beyond the weakening has been shortened or cut off.

In a variation for short feet, the foot plate is preferably not formed across the entire length of the foot, but does not extend at the front past the metatarsal-phalangeal joint. Generally, however, a full-foot version of the foot plate is formed with weakenings as well to allow for a corresponding adaptation to the shape of the shoe and/or the foot.

A further development of the invention provides that, in an orthotic, the foot plate comprises a dimensionally stable section in the area of the foot support and a flexible area in the area of the front edge. The foot plate does not have to be dimensionally stable across the entire length to transfer the forces to be absorbed on the foot or to apply a stabilizing, holding, or supporting momentum on the foot. Rather, it is often enough if increased form stability exists only in the area from the ankle to the metatarsus, so that the heel and the metatarsus are provided with surface support, while an improved foot roll behavior can be achieved with the flexible design in the front area of the foot plate. A flexible section in the area of the front edge is particularly preferable for the wearing in a shoe. Furthermore, in the case of a flexible and possibly elastic design, the front area of the foot plate becomes more pliable and compressible, which makes the foot plate more comfortable to wear.

The weakening or weakenings are preferably formed in the flexible section due to which the foot plate is easier to adapt or shorten as well, for example by passing a knife along the weakening or by using a scissor or another suitable separating tool to cut it.

A floor support may be attached or fixed to the foot support which extends in, on, or below the foot plate so that a two-part design of the foot part with the foot support and the floor plate results. Generally, it is also possible to design the orthotic as one piece, if applicable in an integrally molded or attached foot plate that are joined with each other.

The lower leg support bar may be positioned above a joint and be rotatory around a joint axis relative to the foot support. Alternatively, instead of a joint, the lower leg support bar may be positioned on the foot support and be movable relative to the foot plate by means of a spring, depending on the desired intended use of the orthotic.

The foot plate and/or the foot support and/or the lower leg support part may be provided at least partially with a sheath to improve the wearing properties, provide padding, and an improved visual effect.

An exemplary embodiment of the invention is explained in further detail with the help of the figures provided.

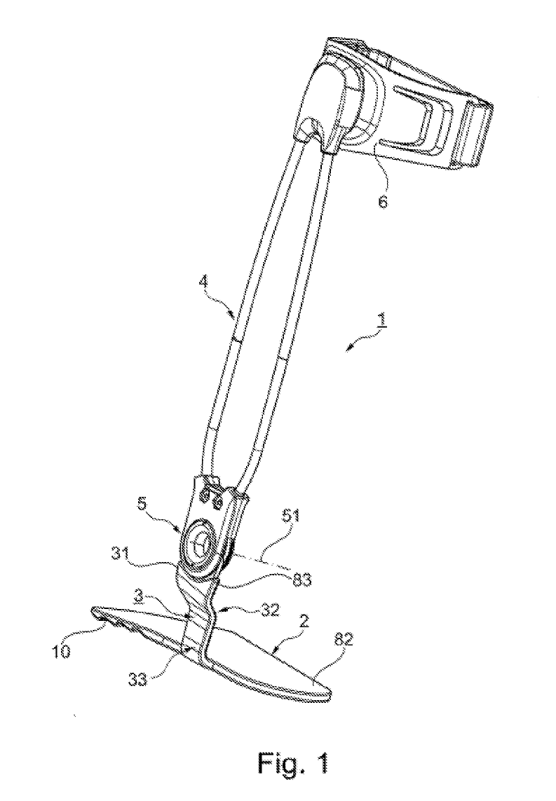

FIG. 1—shows an orthotic as AFO from an overall perspective view;

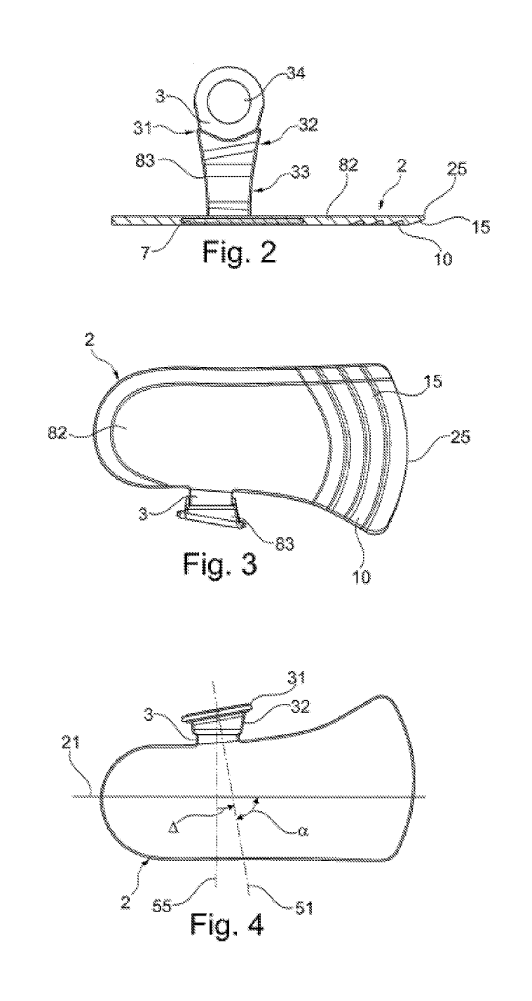

FIG. 2—shows a foot part of the orthotic without lower leg support parts from a lateral view;

FIG. 3—shows a foot part from a bottom view;

FIG. 4—shows a foot part from a top view;

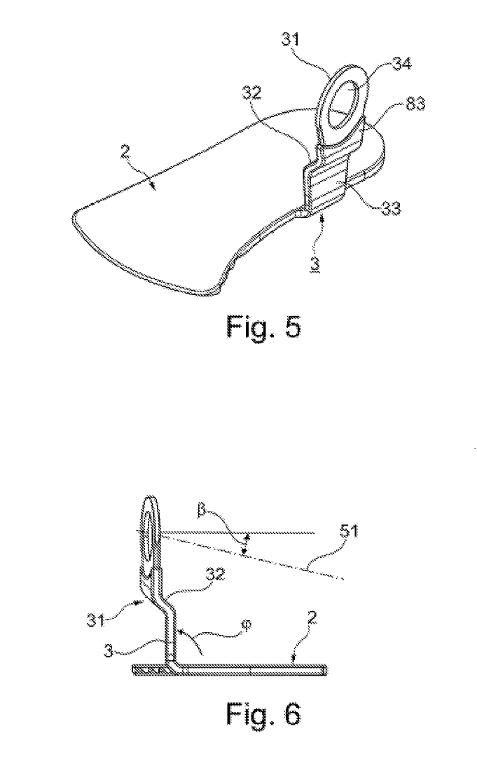

FIG. 5—shows a foot part from a perspective diagonal view;

FIG. 6—shows a foot part from a rear view;

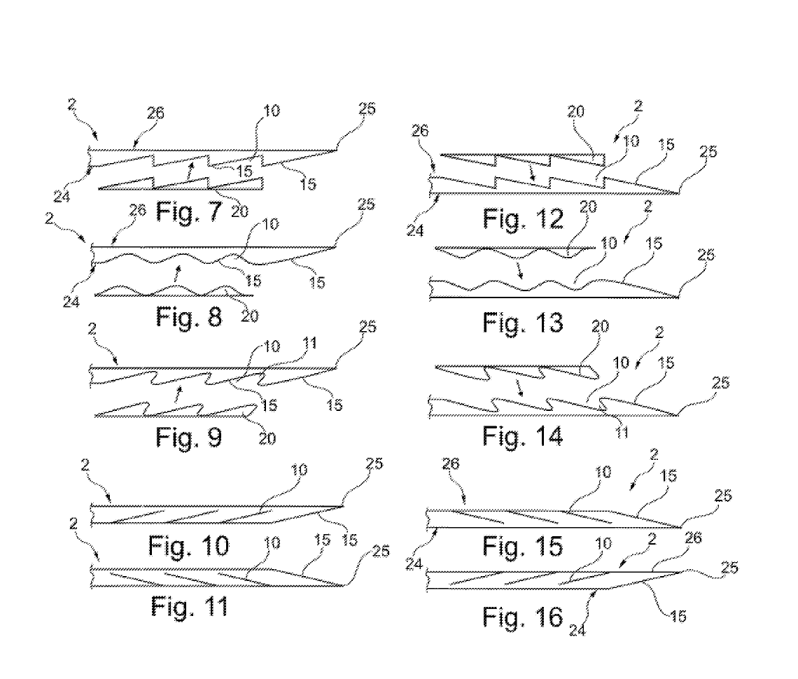

FIGS. 7-11—show details of a foot plate with weakenings on the underside;

FIGS. 12-16—show details of a foot plate with weakenings on the upper side;

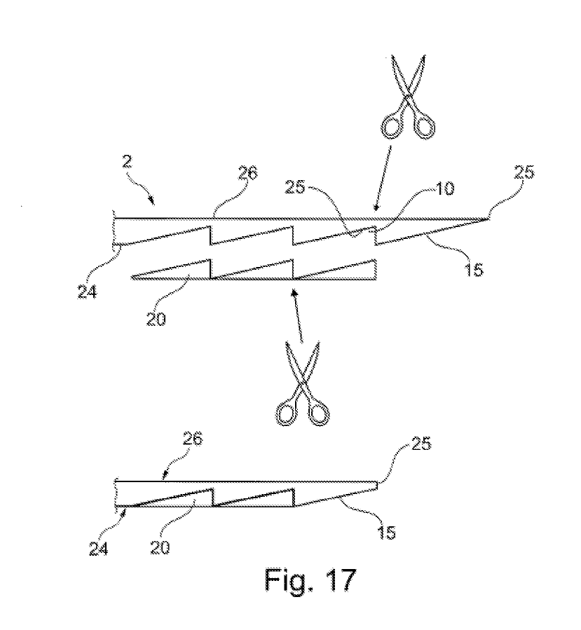

FIG. 17—shows a schematic representation of the adaptation of the length of a foot plate.

FIG. 1 shows an orthotic 1 in the form of an ankle/foot orthotic with a foot plate 2 for supporting a foot that is not shown that protrudes from which a foot support 3 extends in the proximal direction. The foot support 3 is attached to the foot plate 2 at its distal end 33 or formed as one piece together with it. The foot support 3 may be attached by being laminated in, by separate attachment elements such as screws or rivets, by being glued in, snapped in, or by using other form-fit or bonded types of attachment. It is also possible that a floor support 7, which is shown in FIG. 2, is attached to the foot support 3 on the same level as the floor plate 2. It extends into, onto, or below the foot plate 2 and thus forms an angle that allows for a rigid, jointless attachment of the foot support 3 to the foot plate 2.

A joint 5 is attached to the proximal end section 31 of the foot support 3, which is formed as a flat joint and to by means of which a lower leg support bar 4, at whose proximal end a fastening means 6 is arranged so that it can be fixed to a lower leg that is not shown, is rotatably attached to the foot support 3. The fastening means 6 fully surrounds the lower leg and has a cuff with a locking strap. By means of two curved supports of the lower leg support bar 4, the inclination of the upper application point of the lower leg support bar 4 is adjustable relative to the lower leg and/or relative to the orientation of the foot support 3. In addition to the orthotic shown that is used to correct a defective position of the leg, the foot support 3 may be tensioned relative to the lower leg support bar 4 by means of a spring, for example by means of a spring arranged in the joint 5 to provide a dorsiflexion orthotic.

By means of the joint 5, the lower leg support bar 4 is rotatable around a joint axis 51 relative to the foot support 3; a joint rotation around another axis is not provided.

The foot plate 2 comprises a sheathing 82 that may consist of a flexible, potentially elastic material. On the underside of the foot plate 2, the exemplary embodiment shown has three successively arranged weakenings 10 along which the foot plate 2 is easy to shorten. The weakenings 10 have the shape of saw teeth so that, after the material beyond the weakening 10 has been removed, a downwardly inclined or rounded front edge 25 results that makes any rolling off easier and is perceived as less uncomfortable. The foot plate 2 is either shortened or shaped at the narrowest location of the weakening 10 or in the continuation of the inclined or rounded front edge form.

Sheathing 83 is arranged on the foot support 3 as well that extends to the proximal end 31 of the foot support 3 and that only exposes the joint 5 and the adjoining area of the proximal end 31 so that the free rotation of the lower leg support bar relative to the foot support 3 is not impaired. The sheathing 82, 83 improves the wearing comfort because it ensures that the potentially sharp-edged materials of the foot support 3 and/or the foot plate 2 do not come in contact with the body of the orthotic user.

A ledge 32 is formed at the proximal end 31 of the foot support 3 that points away from the foot plate 2 so that the proximal end 31 of the foot support 3 is offset and further away from the foot plate 2 than the distal area 33 of the foot support 3. By means of the ledge 32, the area in which or on which the joint 5 is arranged is distanced from the foot plate 2 so that, if the foot support 3 is medially arranged, the proximal end 31 is medially offset from the foot plate 2. If the foot support 3 is laterally arranged, the ledge 32 and the thus further distance of the proximal end 31 of the foot support 3 relative to a distal end 33 of the foot support, for example in the area of the transition from the foot plate 2 to the foot support 3, makes it possible that the foot support 3 can be guided very close to the osseous structure of the foot without the joint 5 in the area of the ankle rubbing against the osseous ankle protrusion or the contact becoming uncomfortable.

In the exemplary embodiment, the foot support 3 has a distal end 33 that extends away from the foot plate 2 in a substantially straight line. The distal end 33 and the proximal end 31 are offset against each other by the ledge 32 relative to a sagittal plane. At least the material of the distal end 33 of the foot support 3 is flat. In the exemplary embodiment shown, the entire foot support 3 with the distal end 33, the ledge 32 and the proximal end 31 are made from flat material, in particular a metal.

FIG. 2 shows the foot part of the orthotic without the lower leg support bar 4 and without the joint with the foot plate 2 that has a substantially level upper side and an underside with weakenings 10 in the front area. The weakenings 10 are formed like saw teeth and form the intended cutting surfaces along which the foot plate 2 can be shortened and therefore adapted to the different shoe shapes and foot shapes. The weakenings 10 have an inclination or front contour 15 that runs from the front to the back in an obliquely downward direction so that a rolling off is made easier. If the incline ends in a tip, there is no ledge at the front edge of the foot plate 2. If possible, a ledge of less than 2 mm, preferably less than 0.5 mm, is desired so as to not create any interfering influences during the rolling off, even in the sensitive foot area. In the lateral sectional view according to FIG. 2, the foot support 7 can be seen that, in the exemplary embodiment shown, is formed with the foot support 3 and represents a jointless elbow. The foot support 7 is provided with sheathing 82 so that the foot plate 2 is formed from the foot support 7 and the sheathing 82. The sheathing also extends across part of the foot support 3 and is provided there with the reference number 83.

The distal section 33 of the foot support 3 extends substantially vertically upward from the foot plate 2 and is followed by a ledge 32 in which the material of the foot support 3 is bent first outward and then upward so that a joint, which is formed in the exemplary embodiment shown as a round recess 34, or the bearing for the rotatable attachment of the lower leg support bar 4 can be arranged in the proximal end 31.

FIG. 3 shows the foot part according to FIG. 2 from a bottom view. The weakenings 10, in the exemplary embodiment three weakenings 10, are found correspondingly to the front edge 25 of the foot plate 2 at equal distances and across the entire width of the foot plate 2. The foot support 3 has been extruded from the foot plate 2.

FIG. 4 shows a top view of the foot part with the foot plate 2 and the foot support 3 that protrudes vertically from the plane of the page. The foot support 3 is, just as in all other exemplary embodiments shown, arranged medially, approximately in the area of the osseous ankle protrusion of the shin, and extends in a vertical upward direction. A minimum bending radius is provided with which the foot support 3 is connected with the foot plate 2 across the distal area 33 so that the distal area 33 of the foot support 3 can fit as closely as possible against the osseous structure of the foot.

The ledge 32 is asymmetrically formed; the front end of the ledge 32 is bent further in the medial direction than the back end so that, in the top view, a rotation or an angled orientation of the distal end 31 relative to the indicated natural ankle axis 55 results. The joint axis 51 of the joint 5 is oriented at an angle Δ relative to the natural joint axis and, tilted in a horizontal plane toward the front, i.e., in the anterior direction. The angle Δ between the natural joint axis 55 and the joint axis 51 of the joint 5 ranges from 5° to 20°, preferably from 7° and 15°, and particularly preferably 12°, to compensate for an outward rotation while walking or standing. Therefore, the joint axis 51 is oriented in the horizontal plane slanted to the midline 21 of the foot plate 2, which substantially corresponds to the midline of a foot and that runs from the mid-heel area vertical to the ankle joint axis 55. The angle α of the joint axis 51 to the midline of the foot 21 therefore ranges from 85° to 70° and has an anterior orientation; i.e., it is oriented in a forwardly turned manner.

FIG. 5 shows the foot part from a perspective diagonal view that shows that the foot support 3 together with the distal section 33 extends from the foot plate 2 in a substantially vertical manner. The ledge 32 causes the proximal end 31 with the recess 34 as an attachment support for the joint 5 to be oriented in a vertical upward direction as well, but turned forward or in the anterior direction in the longitudinal extension of the foot support 3.

FIG. 6 shows the foot part from a rear view, the angle φ between the foot plate 2 and the distal area of the foot support 3 is 90° in the exemplary embodiment shown, which, in a medial arrangement of the foot support 3, leads to a good fit with the foot bone. An individual adaptation can be performed by means of a plastic deformation of the foot support 3 relative to the foot plate 2, so that the foot support 3 lies as closely as possible against the foot. The distal end 31 of the foot support 3 may, in addition to a rotation around the longitudinal extension, have an inclination in the anterior direction as well so that an angle β is formed between the joint axis 51 and the horizontal shown. Because of this, it is possible to individually adjust the joint axis 51 of the joint that is not shown. The joint axis 51 is preferably positioned so that it coincides with the natural angle axis. A height adjustment may result from an angle adjustment of the foot support 3 by changing the ledge 32 in the form of distancing elements or by inserts or extensions during the attachment of the lower leg support bar 4.

FIG. 7 shows a detail view of a front, anterior end of a foot plate 2 with a front edge 25, an upper side 26, and an underside 24. The front contour 15 slants from the upper side 26 of the forward edge 25 in the direction of the underside 24 in the back so that the foot plate 2 tapers off and ends in the forward anterior direction. This makes it easier to roll off the foot plate 2 and increases the wearing comfort. In the exemplary embodiment shown, three weakenings 10 follow the first contour 15 following the front edge 25. They look like saw teeth and have a corresponding contour 15, i.e., an orientation that slants from the front to the back toward the underside 24. In addition to the foot plate 2, material 20 is prepared that is formed as a strip of material and comprises a contour on the upper side that corresponds to the contour of the foot plate 2 on the underside 24 in the area of the weakenings 10. In the exemplary embodiment shown in FIG. 7, the upper side of the supplementary strip of material has a saw-tooth contour as well with a straight slant from the front edge, i.e., anterior, to the smooth underside, i.e., in the posterior direction. Corresponding to the three weakenings 10 in the foot plate 2, three saw-tooth raises are formed from the additional material 20 and may be arranged or attached, in particular adhered, to complement the contour of the foot plate 2 at its underside 24. The additional material 20 completely fills the weakenings 10 in the exemplary embodiment shown. Generally, it is possible as well that the weakenings 10 are only partially filled. The material 20 may differ from the material of the foot plate 2. It may be of a different color, and it is also possible that the material 20 is made from the same material as the foot plate 2. In the exemplary embodiment shown, the material 20 complements the foot plate 2 in such a way that the underside 24 forms an almost continuous, level surface. The material 20 extends across the entire width of the foot plate 2 and across the length of the weakenings 10 in the foot plate 2. Generally, it is also possible to use the material 20 only partially in the weakenings 10 and to thus not fill the entire underside 24 of the foot plate 2.

FIG. 8 shows a variation of the invention in which, instead of a straight slope, a wave-shaped contour 15 runs as a contour 15 from the front edge 25 to the back end of the foot plate 2. Here as well, three consecutive weakenings 10 located at equal distances from each other are provided into which the supplementary material 20 can be inserted. The upper side of the supplementary material 20 has a shape that corresponds with the weakenings 10 and may be adhered, jammed, or otherwise held in the weakenings 10. If the supplementary material 20 has other mechanical properties, the supplementary material 20 may be used to modify the properties of the foot plate 2, in particular the rolling properties and the comfort.

FIG. 9 shows another variation of the invention with weakenings 10 on the underside of the foot plate 2, in which these form an undercut 11 behind the concave contour 15 that runs obliquely downward and backward. The contour 15 therefore has the shape of an arrow with consecutively arranged undercuts 11 on the underside 24 of the foot plate 2. The undercut 11 makes it possible to provide a form-fit interlocking to prevent the supplementary strip of material 20 from falling out of the weakenings in the direction of the underside 24. By way of an elastic configuration of either the foot plate 2 and/or the supplementary strip of material 20, a jamming and adhering as well as form-fit coupling can be achieved within the weakenings 10. Here as well, the supplementary strip of material 20 complements the foot plate 2 in the area of the weakenings 10 to form a continuous surface with a substantially smooth underside 24.

One variation of the invention is shown in FIG. 10 where a front edge with an obliquely downward contour 15 is formed as well. Instead of the weakening 10 in the form of removed material, the variation of FIG. 10 provides that the weakenings 10 are formed by slits that run substantially parallel to the original contour 15 in the front that obliquely extends from the front edge 25 in the direction of the underside 24. The configuration of the weakenings 10 as slits has the advantage that a substantially even, continuous and smooth underside 24 is created in the applied or inserted state. During a rolling movement, the slits 10 that are the weakenings give in and make it easy to roll off while, at the same time, providing adequate support for forces that apply from the upper side so that the foot plate 2 is still able to support a placed foot.

Another variation of the invention with weakenings 10 on the underside 24 is shown in FIG. 11 in which, however, the course of the front contour 15 is not obliquely downward from anterior to posterior, but obliquely upward. Correspondingly, the weakenings 10 are formed substantially parallel to the front contour 15 and run obliquely upward from the front to the back.

One variation of the invention is shown in FIGS. 12 to 16 which substantially corresponds to the embodiments shown in FIGS. 7 to 11, whereby, however, the foot plate 2 has a closed, substantially smooth underside 24 and whereby the weakenings 10 are formed in the anterior area on the upper side 26. Here as well, the weakenings 10 may be at least partially filled with additional strips of material 20 to provide in the area of the weakenings 10 a smooth, mostly closed surface as well. The design of FIG. 12 corresponds to that of FIG. 7 with the difference that the additional material 20 is placed or set from the top onto the upper side 26 of the foot plate 2 and is fixed in the weakenings 10 or the weakened area. The contour 15 rises from anterior to posterior in the direction from the underside to the upper side, i.e., at an upward slant. The contour of the weakenings 10 corresponds to the contour 15 of the foot plate 2 as it extends from the front edge 25 in the posterior direction.

FIG. 13 shows a wave-shaped design of the weakenings 10 with three consecutively arranged weakenings and an oblique, convex contour 15 that extends from the front edge 25 in the direction of the posterior end of the foot plate 2. The supplementary material 20 completely fills the weakenings 10.

The undercut 11 in the design of the weakenings 10 prevents slippage in the direction of the front edge 25 when additional supplementary material 20 is inserted into the weakenings 10. Furthermore, a distancing vertical to the underside 24 is effectively prevented by a form-fit block.

FIG. 15 shows a design of the weakenings 10 in the form of slits that obliquely extend from the upper side 26 from posterior to anterior in the direction of the underside 24 and therefore run substantially parallel to the front contour 15 that obliquely extends from the front edge 25 in the posterior direction from the underside 24 in the direction of the upper side 26. The slits 10 in the exemplary embodiment shown are not complete; i.e., they do not reach the underside. The same applies to the slits or weakenings according to FIGS. 7 to 14 described above as well. In general, it is possible that the weakenings 10 pass through the foot plate 2 entirely in some areas so that a kind of perforation is formed in the foot plate 2. The upper side 26 or the underside 24 are not completely closed in this case but have recesses, slits, holes, or the like.

FIG. 16 shows a variation of the invention in which, from the front edge 25, the contour 15 obliquely extends from the upper side 26 toward the back, while the weakenings 10 in the form of slits correspondingly obliquely extend from the upper side 26 in the direction of the back lower side. The slits 10 are oriented in a substantially parallel manner to the contour 15 that starts at the front edge 25.

FIG. 17 shows the process of an adaptation of the foot plate 2 to the wishes of a user. First, the anterior front area is cut off in an area of the weakening 10, i.e., in the front area of the weakening, where the contour 15 of the weakening is as close as possible to the upper side 26, by means of scissors. Correspondingly, the first saw-tooth-like area of the supplementary material 20 is cut off in the area of the lowest material strength by means of scissors. This way, the amount of the protrusions of the supplementary material 20 is adapted to the number of the still remaining weakenings 10 in the foot plate 2. In this present exemplary embodiment, only two saw-tooth-like protrusions are present after the supplementary material 20 has been shortened. These may be inserted into the two remaining weakenings 10 at the underside of the foot plate 2.

After having inserted and, if applicable, glued or joined or otherwise fixed the supplementary material 20 in the foot plate 2, the finished foot plate 2 as shown in the illustration below is the result with a new shortened and potentially rounded front edge 25, having possibly a smaller width, an obliquely downward extending contour 25, and a smooth underside 24, because the remaining weakenings 10 of the original foot plate 2 are filled with the supplementary material 20. Consequently, there is now a foot plate 2 with a closed, smooth upper side 26 and a smooth underside 24.

1. A foot plate for supporting a foot, comprising:
a front edge;
at least one weakening which is spaced apart from the front edge and extends in a medial-lateral direction of the foot plate, the at least one weakening comprising:
a first weakening on an underside of the foot plate and having an orientation or rounded contour directed obliquely downward from anterior to posterior;
a second weakening on an upper side of the foot plate and having an orientation or rounded contour directed obliquely upwards from anterior to posterior.

2. The foot plate according to claim 1, wherein the at least one weakening is formed continuously as a broken line or perforation.

3. The foot plate according to claim 1, wherein the at least one weakening extends across the entire width of the foot plate.

4. The foot plate according to claim 1, wherein the at least one weakening follows the contour of the front edge.

5. The foot plate according to claim 1, wherein the at least one weakening is arranged posterior to a metatarsal joint.

6. The foot plate according to claim 1, wherein the at least one weakening includes a plurality of weakenings arranged consecutively at equal distances.

7. The foot plate according to claim 1, wherein the at least one weakening is formed as at least one of a slit, a recess and a reduction in material.

8. The foot plate according to claim 1, further comprising material arranged or arrangeable in the at least one weakening that fills the at least one weakening.

9. The foot plate according to claim 1, wherein the foot plate does not extend past a metatarsal joint of a foot supported by the foot plate.

10. An orthotic comprising:
a foot plate for supporting a foot, the foot plate comprising:
a front edge;
at least one weakening which is spaced apart from the front edge and extends in the medial-lateral direction of the foot plate, the at least one weakening comprising:
a first weakening on an underside of the foot plate and having an orientation or rounded contour directed obliquely downward from anterior to posterior;
a second weakening on an upper side of the foot plate and having an orientation or rounded contour directed obliquely upwards from anterior to posterior
a foot support positioned in the proximal direction of the foot plate;
a lower leg support bar configured to be applied to a lower leg.

11. The orthotic according to claim 10, wherein the foot plate comprises a dimensionally stable section in an area of the foot support and a flexible section in an area of the front edge.

12. The orthotic according to claim 11, wherein the at least one weakening is formed in the flexible section.

13. The orthotic according to claim 10, further comprising a floor support is formed or attached to the foot support that extends in, on, or under the foot plate.

14. The orthotic according to claim 10, wherein the lower leg support bar is positioned rotationally on the foot support above a joint around a joint axis relative to the foot support.

15. The orthotic according to claim 10, wherein the foot support and the foot plate are formed as a single, unitary piece.

16. The orthotic according to claim 1, wherein at least one of the foot plate and the foot support are at least partially provided with sheathing.

17. A foot plate for supporting a foot, comprising:
a front edge;
a plurality of weakenings spaced apart from the front edge and extending in a medial-lateral direction of the foot plate, the plurality of weakenings comprising:
a first weakening positioned on an underside of the foot plate and having an orientation or rounded contour directed obliquely downward from anterior to posterior along a length of the foot plate;
a second weakening positioned on an upper side of the foot plate and having an orientation or rounded contour directed obliquely upwards from anterior to posterior along a length of the foot plate.

18. The foot plate according to claim 17, wherein the plurality of weakenings are formed continuously as a broken line or perforation.

19. The foot plate according to claim 17, wherein the plurality of weakenings extend across the entire width of the foot plate.

20. The foot plate according to claim 17, wherein the plurality of weakenings follow the contour of the front edge.
